(19) United States
(12) Patent Application Publication
(10) Pub. No.: US 2025/0308624 A1
(43) Pub. Date: Oct. 2, 2025

(54) METHOD AND SYSTEM FOR PREDICTING BIOLOGICAL SIGNALING POTENCY AND EFFICACY

(71) Applicants: David Do Le MINH, Mokena, IL (US); David Alvin COOPER, Chicago, IL (US)

(72) Inventors: David Do Le MINH, Mokena, IL (US); David Alvin COOPER, Chicago, IL (US)

(73) Assignee: ILLINOIS INSTITUTE OF TECHNOLOGY, CHICAGO, IL (US)

(21) Appl. No.: 19/095,183

(22) Filed: Mar. 31, 2025

Related U.S. Application Data
(60) Provisional application No. 63/572,522, filed on Apr. 1, 2024.

(51) Int. Cl. G16B 5/00; G16B 40/20

(52) U.S. Cl. CPC G16B 5/00; G16B 40/20

(57) ABSTRACT

A system and method for predicting effects of ligands on receptors. The method includes identifying a plurality of conformations of a receptor, computing a probability of each of the plurality of conformations when the receptor is in an equilibrium complex with a ligand, to obtain a set of equilibrium probabilities, and using the set of equilibrium probabilities to predict an effect of the ligand on the receptor. The system and method can be used for designing drugs to modulate one or more biological signaling pathways, by predicting an efficacy of the ligand for one or more biological signaling pathways, and prioritizing and/or designing ligands based on predicted efficacies.

### CROSS REFERENCE TO RELATED APPLICATION

This application claims the benefit of U.S. Provisional Application Ser. No. 63/572,522, filed on 1 Apr. 2024. The co-pending provisional application is hereby incorporated by reference herein in its entirety and is made a part hereof, including but not limited to those portions which specifically appear hereinafter.

### STATEMENT REGARDING FEDERALLY SPONSORED RESEARCH

This invention was made with government support under 1R01GM127712 awarded by National Institute of Health. The government has certain rights in the invention.

### FIELD OF THE INVENTION

This invention relates generally to methods and a system for predicting effects of a ligand on a receptor and more specifically, for predicting the potency and/or efficacy of biological signaling.

### BACKGROUND OF THE INVENTION

Functional selectivity, also known as biased signaling or biased agonism, refers to the phenomenon in which ligand binding to a single receptor can have different effects on distinct signaling pathways. In the largest class of cell surface receptors, 7 transmembrane receptors (7TMRs), traditionally known as G protein coupled receptors (GPCRs), ligands can differentially activate or inhibit pathways involving heterotrimeric G proteins, 7TMR kinases, and β-arrestins. Signaling efficacy (Eₘₐₓ from concentration-response curves) quantifies the extent of pathway activation at a saturating concentration of ligand. Some 7TMR ligands are balanced, with comparable efficacy for both G protein and β-arrestin pathways; others are biased, with much higher efficacy for a subset of pathways. While 7TMRs are particularly useful targets for drugs (“druggable”), targeted by approximately one third of drugs in the clinic, most of these drugs were designed assuming that they would be balanced. As inappropriate pathway modulation may cause adverse side effects, optimizing functional selectivity is likely to produce safer and more effective drugs targeting 7TMRs and other signaling proteins.

The tragic history of synthetic opioids starkly illustrates the importance of functional selectivity. Fentanyl and its derivatives block pain, exhibiting their analgesic effects by binding to a 7TMR, the opioid receptor (MOR). Although the precise pathways are still debated, adverse side effects of tolerance and respiratory depression are also mediated through the MOR. The medicinal chemists who designed the first synthetic opioids reasoned that compounds with high analgesic potency would be safer than morphine. They touted the high binding affinity of sufentanil to the MOR. Unfortunately, the hypothesis that potent compounds would be safe was incorrect; due to their dangerous side effects, synthetic opioids have become the leading cause of drug overdose deaths in the United States.

Increased recognition of the importance of functional selectivity has inspired extensive research into its mechanisms. One mechanism of functional selectivity is ligand-mediated. This type of functional selectivity is independent of mutation or differential splicing of the receptor or differential expression of transducer elements or downstream effectors. The mechanism of ligand-mediated functional selectivity is generally believed to be stabilization of intracellular pocket conformations that differentially interact with proteins that transduce signals further downstream.

Spectroscopic methods show that different classes of ligands have different effects on 7TMR conformational dynamics. Research has been conducted using double electron-electron resonance spectroscopy to show that ligands with different levels of bias can induce at least four sets of conformations of the angiotensin II type 1 receptor. For the 32 adrenergic receptor, nuclear magnetic resonance (NMR) and single-molecule fluorescence have demonstrated that balanced versus biased ligands have different effects on receptor conformational exchange. Other researchers applied NMR to the MOR to show that biased, unbiased, and partial agonists stabilize different conformations of the receptor. While spectroscopic methods demonstrate the existence of multiple conformations, they have not identified specific three-dimensional structures or determined the extent to which they activate signaling along different pathways.

High-resolution structures provide detailed information about a limited subset of intracellular pocket conformations. X-ray crystallography and cryo-EM structures of 7TMRs are typically solved in complexes that comprise stabilizers, such as antibodies and transducers. These restrict conformational heterogeneity, making structures easy to solve but also obscuring activation mechanisms. MOR structures have been solved as complexes with 17 different ligands with multiple distinct chemical scaffolds and classes of signaling activity. Even though there are a variety of ligands, receptor conformations fall into only two categories: active, for structures complexed to G proteins; and inactive, for structures complexed to antagonists. Presumably the former are capable of G protein signaling while the latter do not activate signaling along any pathway. Two agonist-bound structures of the closely-related 6 opioid receptor may be categorized as intermediate; they feature outward rotations of helix 5 and 6 and inward rotation of helix 7 indicative of 7TMR activation, but the tip of helix 6 is less tilted than in the active structures of the and x opioid receptors. Another 7TMR, the angiotensin II type 1 receptor, has been crystallized in distinct active conformations in complex with balanced versus biased ligands. These notable exceptions show that it is difficult to capture unique intracellular pocket conformations in high-resolution structures of 7TMRs.

Molecular dynamics simulations (MDS) reveal additional 7TMR conformations. Distinct intracellular pocket conformations have been observed in simulations of MOR complexes with a wide variety of ligands. There is a continuing need to use this information for improved drug design.

### SUMMARY OF THE INVENTION

A general object of the invention is to predict effects of a ligand on a receptor, such as modulating biological signaling with a particular potency and efficacy.

Embodiments of the invention include a method for predicting effects of a ligand on a receptor. The method includes identifying a plurality of conformations of a receptor, computing the probability of each of the plurality of conformations when the receptor may be in an equilibrium complex with a ligand, and using the set of the equilibrium probabilities to predict the effect of the ligand on the receptor. A machine leaning model is provided to group configurations from MDS into conformations with model outputs as signaling efficacies along different pathways.

In embodiments, the effect of a ligand may modulate biological signaling with a predicted potency and efficacy. In some embodiments, the ligand may be a positive or negative allosteric modulator that alters biological signaling of another ligand that binds to the orthosteric site of a receptor. The effect of a ligand may modulate the rate of enzyme catalysis. Embodiments also include identifying the plurality of conformations which include performing molecular dynamics simulations (MDS) of the receptor complexed with different ligands to obtain a plurality of configurations.

Embodiments also include identifying a plurality of conformations, and clustering sampled receptor configurations into the conformations. Desirably the clustering is performed with parameters such that the same conformations may be observed with different ligands. Embodiments also include equilibrium probabilities that may be calculated based on the fraction of simulations of a receptor-ligand complex.

In embodiments, predictions are based on inputting equilibrium probabilities of conformations into a machine learning model. In embodiments, the machine learning model may be based on multiple linear regression (MLR). The machine learning model may be trained and cross-validated by providing inputs and outputs for a set of ligands for which the effect has been experimentally measured.

In embodiments, the training and cross-validating may be performed by leave-one-out procedures. In some embodiments, the test set may include one sample and the training set may include the remainder of the data. In some embodiments, the machine learning model may be trained based on a leave-one-out loss function. In some embodiments, each training set may be divided into a sub-test set and a sub-training set.

In embodiments, the sub-test set may include one sample and the sub-training set may include the remainder of training data. In some embodiments, the leave-one-out loss function may include the mean square error of the sub-test set, averaged over all the sub-training processes for the training set.

Embodiments include optimizing hyperparameters for clustering and parameters for machine learning to minimize a loss function. In some embodiments, the hyperparameters for clustering include the number of hierarchical clusters, the delta value for conversion between the distance matrix and similarity matrix, and the number of conformations returned from spectral clustering and multiple linear regression slopes.

In embodiments of the invention, the method includes the step of cross-validating the computational system by splitting the data into a training set and a test set and assessing the quality of effect prediction on the test set.

Also included is a method for designing ligands to modulate one or more biological signaling pathways, including the steps of predicting the efficacy of a ligand for one or more biological signaling pathways using the method and designing ligands based on the predicted efficacies. Embodiments may also include a method for prioritizing the experimental synthesis and characterization of new ligands, including the steps of predicting the efficacy of ligands for one or more biological signaling pathways using the method and prioritizing ligands based on their predicted efficacies.

The invention also includes a method for designing drugs to modulate one or more biological signaling pathways, including the steps of docking ligands to representative structures from conformations identified through the machine learning model. Embodiments may also include a method of sampling binding pocket conformations by applying biasing potentials to the representative conformations, on other parts of the receptor, and docking ligands to the sampled binding pocket conformations.

Embodiments of the invention also include a method for identifying structural features associated with activating one or more biological signaling pathways, which may include the steps of weighing conformation-specific histograms by parameters from the machine learning model. Embodiments may use a general activation function, nonzero when the weighted histograms have the same sign (Equation 8), or a selective activation function, nonzero when the weighted histograms have the opposite sign (Equation 9). The general and selective activation scores may be numerical integrals of the respective activation functions and may be used to rank the relevance of structural features.

The invention further includes a computational system for predicting biological signaling efficacy, including one or more processors configured to receive samples from a configurational distribution of a receptor-ligand complex as input. Embodiments include one or more machine learning models within the processor, trained to process the equilibrium probability of each conformation and generate predictions of efficacy for one or more biological signaling pathways as output.

In embodiments, the samples may be obtained through molecular dynamics simulations based on standard atomistic force fields and simulation settings mirroring experimental conditions for which a prediction may be desired. In embodiments, the simulation settings may include temperature, pressure, and ionic concentrations. Simulations may also include one or more different ligands bound to the receptor at different locations.

In embodiments, the system and its processors may be further configured to use a clustering module for categorizing sampled receptor configurations into conformations. In embodiments, the clustering module may include one or more parameters adjustable for the observation of the same conformations across various ligands. In embodiments, the processors further develop a continuous function, eliminating the need for using conformations as an intermediate, configured to map directly from configurations of the receptor-ligand complex to efficacy predictions for one or more biological signaling pathways.

In embodiments, the predictions may include specifying the impact of each conformation on the efficacy of one or more biological signaling pathways. In embodiments, the computational system may include biasing potentials during simulations, with mechanisms for resampling or reweighting to remove their effects.

In embodiments, the system and its processors may be further configured for training and validating the system prior to use. In embodiments, the training may include optimizing hyperparameters for a clustering process and a machine learning model within the computational system to maximize the accuracy of efficacy prediction using inputs and outputs for a set of ligands for which the efficacy has been experimentally measured. In embodiments, the validating may be performed by splitting the data into training and test sets and assessing the quality of efficacy prediction in the test sets.

### BRIEF DESCRIPTION OF THE FIGURES

FIG. 1 is a flowchart conceptually illustrating a method for predicting effects of a ligand on a receptor, according to embodiments of the invention.

FIG. 2 is a flowchart illustrating a method according to embodiments of the invention.

FIG. 3A illustrates an example of signaling efficacy predictions of ligands for G protein.

FIG. 3B illustrates an example of signaling efficacy predictions of ligands for β-arrestin-2 pathways.

FIG. 4 illustrates an example of multiple linear regression slopes of each conformation, according to embodiments of the invention.

FIGS. 5A and 5B illustrate an example of alpha carbon distances with the largest general and selective activation scores, according to embodiments of the invention.

FIGS. 6A-D illustrate an example of amino acid positions containing dihedral angles with the highest general and selective activation scores.

FIGS. 7A and 7B illustrate an example of distances in the sodium binding pocket with the highest general and selective activation scores, according to embodiments of the invention.

FIG. 8 illustrates an example of the percentages of simulations of complexes with different ligands in each conformation, according to embodiments of the invention.

FIG. 9 illustrates another example of percentage of conformations accessed in simulations with each complex. Abbreviations: DAM: DAMGO, FEN: fentanyl, LFT: lofentanil, MPH: morphine, TRV: TRV130, SR: SR17018, PZM: PZM21, FH: FH210, C5: c5guano, C6: c6guano, MP: mitragynine pseudoindoxyl, APO: apo.

FIGS. 10A and 10B illustrate an example of efficacy response functions of the distance between sodium pocket polar atoms: (FIG. 10A) OD1 of D116^{2.50} and ND2 of N334⁷⁻⁴⁹ and (FIG. 10B) OD2 of D116^{2.50} and ND2 of N330⁷⁻⁴⁵. The curves correspond to the G protein and β-arrestin-2 efficacy response functions (ERF), respectively.

FIGS. 11A-C illustrate an example of representatives of features used to discretize configurations into conformations: FIG. 11A) dihedral angles, FIG. 11B) alpha carbon distances, and FIG. 11C) distances between polar atoms in the intracellular region of the MOR.

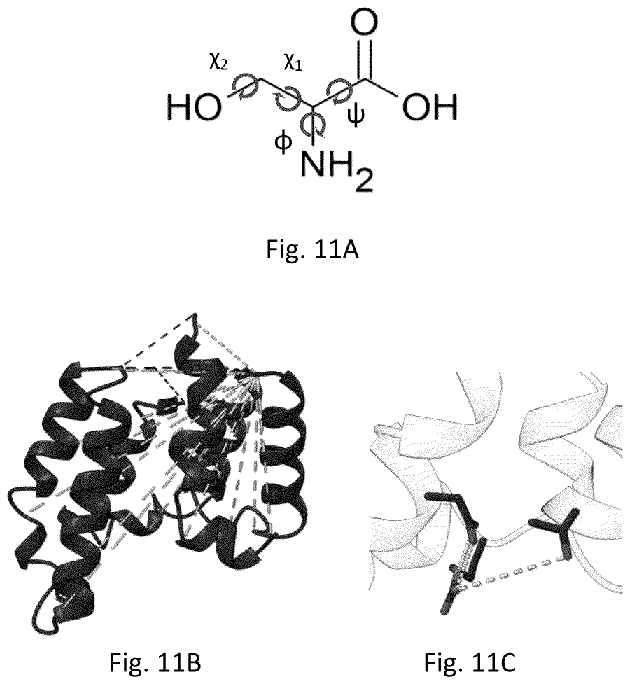

### DETAILED DESCRIPTION OF THE INVENTION

The invention includes a method and system for predicting effects of a ligand on a receptor. The method generally includes a step of identifying a plurality of conformations of a receptor. Embodiments may also include computing the probability of each of the plurality of conformations when the receptor may be in an equilibrium complex with a ligand. Embodiments also include using the set of the equilibrium probabilities to predict the effect of the ligand on the receptor. The invention is desirably implemented with a computer system including one or more data processors in combination with at least one non-transitory recordable medium including a set of encoded software instructions to perform the method steps, such as those described hereafter.

Embodiments of this invention includes a computational device or model that connects conformational equilibria to functional selectivity. Signaling efficacy can be linearly proportional to the equilibrium population of intracellular pocket conformations. Equilibrium populations are used to accurately estimate by, for example, molecular dynamics simulations. Suitable definitions of these intracellular pocket conformations are determined by training a machine learning model. Intracellular pocket conformations have a broad range of signaling efficacy along different pathways. Efficacy response functions and activation scores are effective metrics for identifying structural features associated with general and selective activation. These analyses lead to predicted structural mechanisms for general and selective activation that are supported by previous computational and experimental studies.

In general, the input to the computational device is a set of samples from the configurational distribution of a receptor-ligand complex. The samples may be from a molecular dynamics simulation based on standard atomistic force fields and performed using established packages. Simulation settings, such as temperature, pressure, and ionic concentrations, should be similar to experimental settings for which a prediction is desired. Simulations may be performed with biasing potentials whose effects should be removed by resampling or reweighting.

The invention clusters the sampled receptor configurations into conformations. Generally speaking, configurations are three-dimensional coordinates of each atom in the system and exist in a continuous space. Conformations are groups of similar configurations and are discrete. Clustering discretizes the continuous space. Clustering desirably is performed with parameters such that the same conformations are observed with different ligands.

Once configurations are clustered into conformations, the fraction of configurations in each conformation is input into a machine learning model. An output of the computational device can be the efficacy of the ligand for one or more biological signaling pathways. The efficacy may be reported as a ratio of efficacies relative to a reference compound.

The system is desirably trained and validated. Training requires inputs and outputs for a set of ligands for which the efficacy has been experimentally measured. Hyperparameters for the clustering process and machine learning model are optimized to maximize the accuracy of efficacy prediction. Validation can be performed by splitting the data into training and test sets and assessing the quality of efficacy prediction in the test sets.

In embodiments, it is also possible to develop a continuous function that maps from configurations to efficacy scores without using conformations as an intermediate.

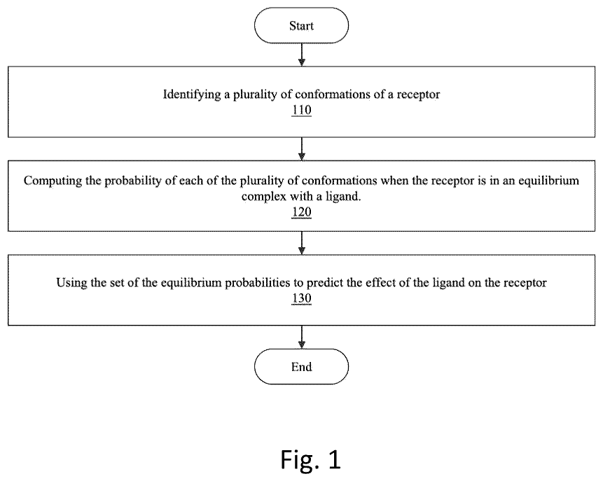

FIG. 1 is a flowchart that describes a method for predicting effects of a ligand on a receptor, according to embodiments of the invention. In embodiments, at 110, the method includes identifying a plurality of conformations of a receptor. At 120, the method includes computing the probability of each of the plurality of conformations when the receptor is in an equilibrium complex with a ligand. At 130, the method includes using the set of the equilibrium probabilities to predict the effect of the ligand on the receptor.

Embodiments of the invention include a machine learning model, such as based upon the hypothesis that signaling through transmembrane receptors is linearly proportional to the equilibrium probability of observing intracellular pocket conformations. Model inputs are molecular simulations of the opioid receptor in complex with different ligands. For eleven ligands, the model calculates the efficacy of G protein and β-arrestin-2 signaling to within 8.5% and 18.4% of experiment, respectively. Structural features that the model associates with activation are intracellular pocket expansion, toggle switch rotation, and sodium binding pocket collapse. Distinct pathways are activated by different arrangements of the ligand and sodium binding pockets and the intracellular pocket.

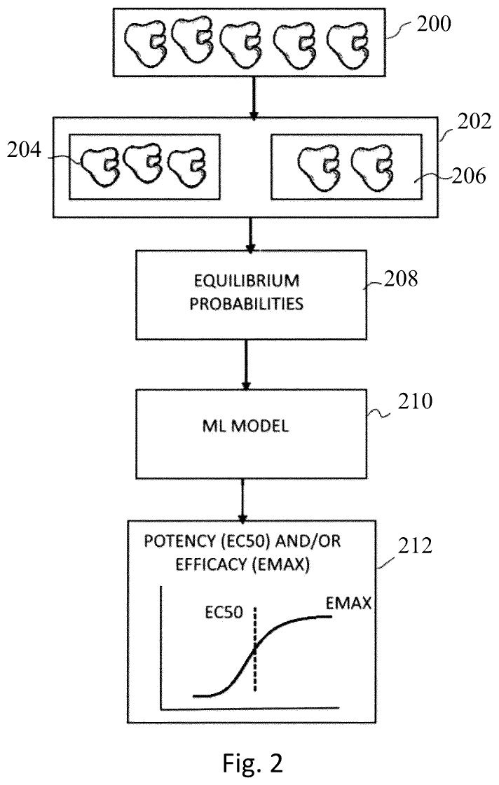

FIG. 2 is a flowchart that describes a method, performed by one or more computers, for predicting effects of a ligand on a receptor, according to presently preferred embodiments of the invention. Exemplary predicted effects include the ligand's ability to modulate biological signaling with a predicted potency and/or efficacy, and/or to modulate a rate of enzyme catalysis.

Step 200 includes simulations (e.g., molecular dynamics simulations) of a receptor complexed with different ligands to obtain a plurality of configurations. Step 202 includes clustering sampled receptor configurations into conformations, performed with parameters such that same conformations are observed with different ligands. Only two clusters 204 and 206 are shown for illustration purposes, and the number of actual clusters, and the number of configurations per cluster, will vary.

Step 208 includes computing a probability of each of the plurality of conformations when the receptor is in an equilibrium complex with a ligand, to obtain a set of equilibrium probabilities. As used herein “equilibrium probabilities” generally refer to the probabilities of observing conformations of the receptor-ligand complex at a specified thermodynamic state once they are in equilibrium, not changing over time. In embodiments, equilibrium probabilities are calculated based on the fraction of configurations in simulations of a receptor-ligand complex in which a particular receptor conformation is sampled. In embodiments, the receptor-ligand complex is simulated in explicit membrane and water with the thermodynamic state defined by setting the temperature, volume, and number of particles to model the conditions of functional assays. Step 210 uses the set of equilibrium probabilities to predict an effect of the ligand on the receptor. For example, predicting the effect of the ligand on the receptor can include inputting equilibrium probabilities of conformations into a machine learning model. In embodiments, the machine learning model is based on multiple linear regression. The resulting effect, such as potency and/or efficacy is provided in step 212.

The machine learning model of step 210 is or has been desirably trained and cross-validated by providing inputs and outputs for a set of ligands for which the effect has been experimentally measured. The training and cross-validating can be performed by leave-one-out procedures, e.g., a test set including one sample and a training set being a remainder of set data. As an example, the machine learning model can be trained based on a leave-one-out loss function, wherein each training set is divided into a sub-test set and a sub-training set. The sub-test set can include one sample and the sub-training set is the remainder of data. The leave-one-out loss function can also include a mean square error of the sub-test set, averaged over all sub-training processes for the training set.

In embodiments, the training optimizes hyperparameters for clustering and parameters for machine learning to minimize a loss function. The hyperparameters for clustering can be a number of hierarchical clusters, a delta value for conversion between a distance matrix and a similarity matrix conversion, and a number of conformations returned from spectral clustering and the parameters for machine learning include multiple linear regression slopes.

The training can also include cross-validating the computational system by splitting the data into a training set and a test set and assessing a quality of effect prediction in the test set.

The discussion below include examples of materials and methods used in embodiments of the invention.

### Molecular Dynamics Simulation

Three-dimensional models of human MOR were built in the apo form and bound to various ligands based on experimental structures available in the Protein Data Bank (Table 1). Models were built based on the first chain of the 7TMR that appears in each file, excluding additional 7TMR subunits and G proteins. The apo structure was based on the DAMGO-bound structure 8EFQ with DAMGO removed. Proteins were protonated with pdb2pgr (version 3.6.1) with a pH of 7.0. Ligands were protonated using RDKit (version 2023.03.1) with a pH of 7.0. Protein-ligand complexes were solvated with 0.15 M NaCl and inserted into a membrane using the custom scripts (https://github.com/swillow/pdb2amber). The scripts build a DPPE lipid bilayer around the protein after alpha carbon alignment to a MOR structure (5C1M) in the Orientations of Proteins in Membranes database. Complexes were parameterized using AMBER forcefields ff14SB for the protein, opc3 for the water, and lipid17 for the membrane. Ligands were parameterized using the GAFF2 force field from AmberTools (version 22.0).

MDS were performed using OpenMM version 8.0.0. The systems were minimized using the local energy minimizer simtk.openmm.app.simulation.Simulation.minimizeEnergy with 500 kJ/mol/nm2 restraints on the protein and membrane, 5000 iterations, and a tolerance of 100 kJ/mol. Equilibration was performed in several stages. First, water and membrane were equilibrated with 500 picoseconds of NVT simulation with 300 kJ/mol/nm2 restraints on the protein and z-coordinate positions of the membrane. Next, two cycles of 5 nanosecond NPT simulation were performed, with the first cycle using a Monte Carlo Membrane Barostat and the second using a Monte Carlo Barostat. All equilibration simulation was performed with a time second of 2 femtoseconds at 300 K. Integration moves were made using the Langevin Middle Integrator.

Production simulations were performed in triplicate for 500 ns each with a timestep of 3 femtoseconds, saving configurations every 7.5 picoseconds. Production runs were performed at 300 K and 1 bar of pressure with a Monte Carlo Barostat and powered by the Langevin Middle Integrator. Calculations were performed using computing resources provided by the Advanced Cyberinfrastructure Coordination Ecosystem: Services & Support (ACCESS) program.

### Efficacy Calculations

A machine learning model was built to compute signaling efficacies. Model inputs were configurations from MDS of complexes with each ligand. Outputs were experimental efficacies curated from the literature (Tables 1 and 2). Experimental data from the cyclic adenosine monophosphate (cAMP) assay, which measures the inhibition of downstream cAMP production, were used for G protein signaling efficacies. For the β-arrestin-2 pathway, data from standard assays for measuring β-arrestin-2 recruitment were included: NanoBit, BRET, PathHunter, and Tango. Assays performed with G protein receptor kinases (GRKs) were excluded.

The model is particularly simple and interpretable, based on multiple linear regression (MLR). First, configurations from MDS are clustered into C conformations. This step yields f_{l,c}, the fraction of simulations with ligand l in conformation c. Next, the signaling efficacy is computed based on a weighted sum over all conformations,

$${E}_{l , G} = {β}_{G , 0} + {\sum }_{c = 1}^{C} {β}_{G , c} {f}_{l , c} , \qquad ( 1 )$$

where E_{l,G} is the signaling efficacy of ligand l and β_{G,n} are regression slopes along G protein pathway. Analogous terms for the β-arrestin-2 pathway, E_{l,β} and β_{β,n}, are computed via an analogous expression. The MLR implementation is used in the open source python package scikit-learn (version 1.3.0).

The machine learning model has parameters and hyperparameters. The parameters are regression slopes. Once conformations are defined and fractions computed, these are uniquely defined by the least squares solution for a particular set of input populations and output efficacies. However, the process of defining conformations involves hyperparameters (1) for distances between configurations and (2) for clustering.

### Distances Between Configurations

Each sampled configuration was characterized using three vectors of features: \vec{θ}, the N_{θ} backbone and side chain torsion angles (ψ, ϕ, χ₁, and χ₂ angles) of all the intracellular protein residues (G84^{1.46}-D116^{2.50}, S156^{3.39}-W194^{4.50}, P246^{5.50}-F291^{6.44}, Y328^{7.43}F349^{H8}); \vec{C}, the N_{C} pairwise distances between alpha carbons from residues in the middle and intracellular end of each helix, with at least one residue in between, and the middle of each intracellular loop (G84^{1.46}, V91^{1.53}, T99^{ICL1}, K102^{ICL1}, T105^{2.39}, D116^{2.50}, S156^{3.39}, S164^{3.47}, A170^{3.53}, L178^{ICL2}, P183^{4.39}, W194^{4.53}, C253^{5.57}, R260^{5.64}, L267^{ICL3}, K271^{6.11}, M283^{6.23}, F291^{6.30}, Y328^{7.43}, Y338^{7.53}, G343^{H8}, and F349^{H8}); and \vec{H}, the N_{H} distances between intracellular hydrogen bond donors and acceptors observed to be within 8.0 Å of the first configuration of FH210-bound complex (PDB ID 7SCG). For each vector, the subscript k was used as an index. The distance between configurations i and j was defined as,

$${D}_{Θ} ( i , j ) = \sqrt{\frac{1}{{N}_{θ}} {{\sum }_{k = 1}^{{N}_{θ}} [ 1 \; 80 - \vert \vert {θ}_{k} ( i ) - {θ}_{k} ( j ) ) \vert - 180 \vert ]}^{2}} \qquad ( 2 )$$

$${D}_{C} ( i , j ) = \sqrt{\frac{1}{{N}_{C}} {{\sum }_{k = 1}^{{N}_{C}} [ {C}_{k} ( i ) - {C}_{k} ( j ) ]}^{2}} , \qquad ( 3 )$$

$${D}_{H} ( i , j ) = \sqrt{\frac{1}{{N}_{H}} {{\sum }_{k = 1}^{{N}_{H}} [ {H}_{k} ( i ) - {H}_{k} ( j ) ]}^{2}} , \qquad ( 4 )$$

$${D}_{ij} = {w}_{θ} \frac{{D}_{θ} ( i ,}{max ( {D}_{θ} ( i , j ) )} + {w}_{C} \frac{{D}_{C} ( i , j )}{max ( {D}_{C} ( i , j ) )} + {w}_{H} \frac{{D}_{H} ( i , j )}{max ( {D}_{H} ( i , j ) )} . \qquad ( 5 )$$

Equation 2 is based on the smallest difference, accounting for periodicity, between torsion angles. Equations 2, 3 and 4 demonstrate how each component was calculated, while equation 5 shows how components are combined to represent a single value for the distance between frames i and j. The sum of weights was constrained to one, such that w_{θ}+w_{C}+w_{H}=1.

### (2) Clustering

Intracellular conformations were defined by clustering. First, hierarchical clustering with complete linkage was performed using SciPy (version 1.11.3) based on pairwise distances calculated from equation 5, leading to H hierarchical clusters. Second, the pairwise RMSD between the hierarchical cluster centroids was calculated. The RMSD distance matrix of centroids D_{rmsd} was converted into a similarity matrix S_{rmsd} using a Gaussian function implemented in scikit-learn,

$${S}_{rmsd} = {e}^{\frac{- {( {D}_{rmsd} )}^{2}}{2 {δ}^{2}}} , \qquad ( 6 )$$

which introduces a bandwidth hyperparameter 6. Centroids and corresponding configurations were grouped into conformations using spectral clustering from scikit-learn with the cluster_qr assignment algorithm. This algorithm uses S_{rmsd} and returns C conformations.

### Training and Cross-Validation

Leave-one-out procedures were used both for training and cross-validation. In statistics, cross-validation involves separating the data into a training set and a test set. A machine learning model that does not overfit the data produces accurate outputs not only for the training set, but also for the test set. In leave-one-out cross validation, the test set comprises of one sample and the training set is the remainder of the data. The training is repeated multiple times with each sample as the test set.

The machine learning model was trained based on a leave-one-out loss function. Each training set was further divided into a sub-test set comprising one sample and sub-training set containing the remainder of the data. Sub-training was repeated multiple times with each training set sample as the sub-test set. The loss function was the mean square error of the sub-test set, averaged over all the sub-training processes for the given training set.

The loss function was optimized via a grid search. Distance components were weighted according to (w_{θ}, w_{C}, w_{H})∈{(w, w, 1−2w), (w, 1−2w, w), (1−2w, w)}, where w∈{0.1, 0.2, 0.25, 0.33, 1}. Clustering hyperparameters were varied over a range of integers, with the number of clusters H between 2 and 40, the bandwidth 6 between 1 and 3, and the number of conformations C∈{2, 3, . . . , H−1}. For each combination of hyperparameters, the leave-one-out loss is computed for both G protein and β-arrestin-2 efficacy. Hyperparameters were selected based on minimizing the sum of the leave-out-out loss of both efficacies.

Leave-one-out training led to consistent values of all machine learning hyperparameters. For all 11 models trained with each ligand as the test set, the best performance was observed with the distance weights of w_{θ}=0.25, w_{C}=0.25, and w_{H}=0.5, the number of hierarchical clusters H=40, and bandwidth δ=2, and the number of conformations C=14. The consistency of these parameters indicates that the training procedure is robust, insensitive to the inclusion or exclusion of any single ligand. Thus, these hyperparameters were used for all models reported in the paper.

The machine learning model was validated via leave-one-out cross-validation. Reported signaling efficacies and performance metrics are based on these models which do not include test compounds within training sets. This procedure is consistent with the way efficacy would be predicted for a ligand with a known binding pose but unknown efficacy.

### Structural Analysis

To understand the relationship between structural features and receptor activation, an efficacy response function (ERF) was defined based on the defined conformations and MLR weights from the machine learning model. First, slopes are recorded from each training iteration of the single conformational space that minimizes the loss function. For each structural feature, a kernel density estimate (KDE) of the probability density function within each conformation is computed using numpy.histogram, with the density feature (version 1.26.0). Each torsional sample Φ was triplicated at Φ+2π and Φ−2π to make the density estimate continuous over the period.

The KDE was normalized based on evaluating the integral over the range [−π, π] using numpy.histogram, with the density feature (version 1.26.0). Next, the normalized KDEs were multiplied by corresponding mean MLR slopes and summed together, resulting in efficacy response functions. ERFs were computed for all features that were used to compute distances (see Distances between configurations) and for both G protein and β-arrestin-2 activation. ERFs calculations were extended to residues in the binding pocket, which did not contribute to distances, and weighted with the corresponding intracellular configuration.

An ERF helps identify changes in the probability density that favor an activation process. It is important to note that an ERF is not a probability density function. Because MLR slopes may be negative, an ERF can be negative. Moreover, they are not normalized. Nonetheless, ERFs are helpful for interpreting the machine learning model. Shifting a probability density towards a region with high ERF values favors activation. Conversely, shifting a probability density towards a region with low ERF values favors inactivation. If the ERF is near zero over the entire range, the feature is unrelated to activation.

As many ERFs were computed, several metrics were defined to help prioritize, in an unbiased way, which structural features to focus the attention on. The general activation function a(x) is:

$$a ( x ) = \{ \begin{matrix}min ( \vert {r}_{G} ( x ) \vert , \vert {r}_{β} ( x ) \vert ) \\ 0 otherwise\end{matrix} \qquad ( 8 )$$

$$if ( {r}_{G} ( x ) > 0 and {r}_{β} ( x ) > 0 ) or ( {r}_{G} ( x ) < 0 and {r}_{β} ( x ) < 0 ) ,$$

where r_{G}(x) and r_{β} are ERFs for the G protein and β-arrestin-2 pathways, respectively. This function is nonzero in regions where ERFs of both pathways have the same sign. Conversely, the selective activation function is:

$$s ( x ) = \{ \begin{matrix}\vert {r}_{G} ( x ) \vert - \vert {r}_{β} ( x ) \vert \\ 0 otherwise\end{matrix} \qquad ( 9 )$$

$$if ( {r}_{G} ( x ) > 0 and {r}_{β} ( x ) < 0 ) or ( {r}_{G} ( x ) < 0 and {r}_{β} ( x ) > 0 ) .$$

This function is nonzero in regions where ERFs of both pathways have opposite signs. For each function, corresponding scores were also defined as a numerical integral over the domain: the sum of the function was computed 1,000 evenly spaced points over the domain and multiplied by the spacing.

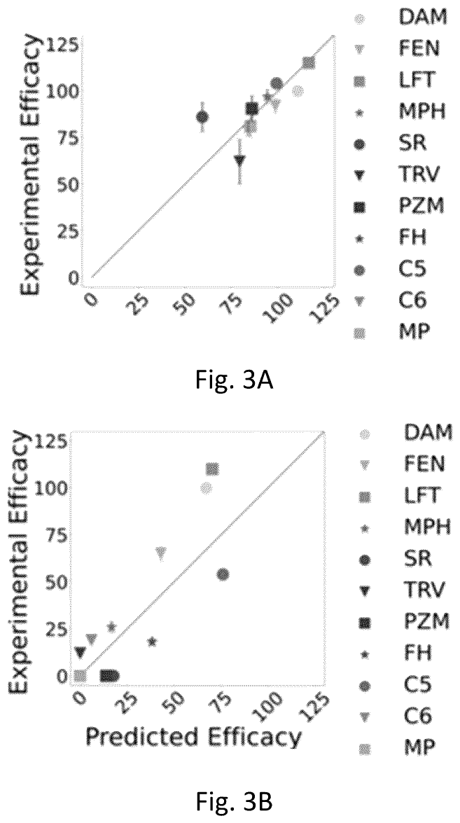

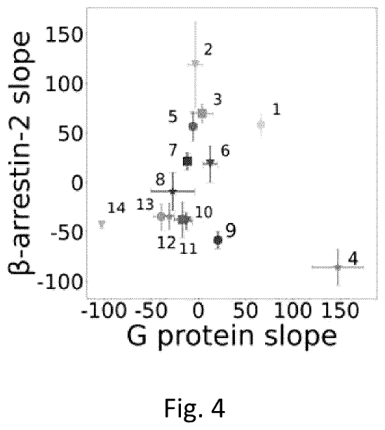

FIG. 3 illustrates the accuracy of the model supporting the hypothesis that signaling efficacy is proportional to the equilibrium probability of observing intracellular pocket conformations. The model could be improved with a larger and more consistent training set and extended to compute other properties. While additional ligands could lead induce additional conformations, they could also lead to more precise regression slopes. Due to limited availability, not all efficacy data were based on the same assays (Table 2). A model trained on consistent data is expected lead to more accurate efficacy predictions. Other properties that may be proportional to equilibrium populations of intracellular pocket conformations include efficacies of Ga subtypes and parameters of the Black-Leff operational model: the transducer ratio and dissociation constant of the agonist-receptor-transducer complex.

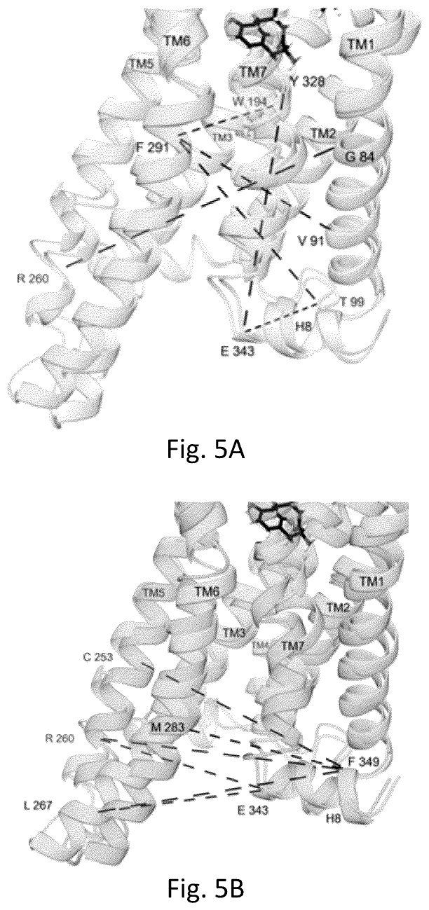

The results also affirm that many intracellular pocket conformations defy simple classification. Conformations are not simply active or inactive. Neither are they simply biased towards G protein or β arrestin signaling. Instead, conformations have a broad range of signaling efficacy across multiple pathways (FIG. 4).

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

### Example 1

Model predictions of Eₘₐₓ are accurate and precise (FIG. 3). Compared to median experimental values, the efficacy of G protein and β-arrestin-2 pathways is predicted with a mean absolute error of 8.1% and 18.4% and a root mean squared error of 10.8% and 21.2%, respectively. The coefficients of determination (R²) are 0.67 and 0.69, respectively. The standard deviation of efficacy predictions is small, a demonstration that the training procedure is robust.

FIG. 3 illustrates an example of signaling efficacy predictions of ligands for (A) G protein and (B) β-arrestin-2 pathways. On the x axis, each predicted efficacy (relative to DAMGO) is based on a model trained using all other ligands. The error bar is the standard deviation across 11 cross validation models. On the y axis, each experimental efficacy is the median of reported values listed in Table Sl. Error bars are standard deviations of these values. Abbreviations: DAM: DAMGO, FEN: fentanyl, LFT: lofentanil, MPH: morphine, TRV: TRV130, SR: SR17018, PZM: PZM21, FH: FH210, C5: c5guano, C6: c6guano, MP: mitragynine pseudoindoxyl. Efficacies are percentages relative to DAMGO.

The model is based on 14 conformations with a wide range of activity (FIG. 4). These conformations are indexed in decreasing order by average regression slope along G protein and β-arrestin-2 pathways; conformation 1 has the largest average slope and conformation 14 the smallest. However, the average oversimplifies the activity of these conformations. Conformations 4 and 9 promote G protein signaling and repress β-arrestin-2 signaling. Conformations 2, 3, 5, and 7 promote β-arrestin-2 signaling at different levels but have minimal effect on G protein signaling. Conformations 1 and 6 recruit both G proteins and β-arrestin-2. The remaining conformations repress signaling.

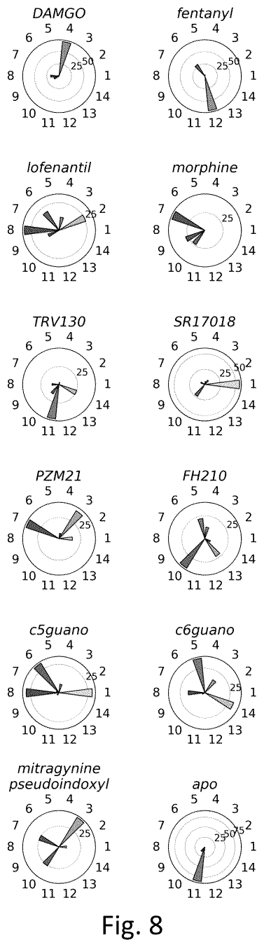

Simulations with different ligands access different ratios of these shared conformations (FIG. 8). Some simulations, such as the apo system (conformation 11) and the complex with DAMGO (conformation 4), are dominated by a single conformation. Others, such as simulations of complexes with PZM21, c5guano, and c6guano, are spread across two or more conformations. Complexes with comparable ligands primarily access distinct conformations, explaining differences in efficacy. Complexes with fentanyl and lofentanil both access conformation 6, but it is not the most populated conformation of either. Similarly, conformation 8 is shared between complexes with both c5guano and c6guano, but both complexes are dominated by other conformations.

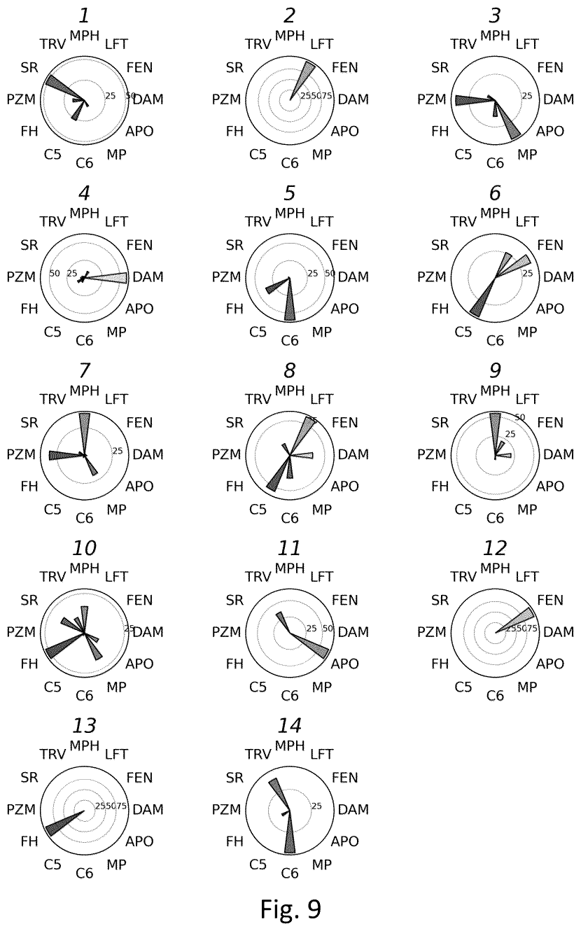

Most conformations are accessed in simulations with multiple ligands (FIG. 9). However, there are three conformations that are unique to complexes with a specific ligand: 2 (lofentanil), 12 (fentanyl), 13 (FH210).

### Example 2

G Protein and β-Arrestin-2 Activation are Associated with Distinct Structural Changes

Several functions are defined to understand the relationship between structural features and receptor activation. The efficacy response function (ERF) is a sum of estimated probability density functions of a structural feature weighted by linear regression slopes. The general and selective activation scores quantify whether the ERF is significant in both or only one of G protein and β-arrestin-2 recruitment.

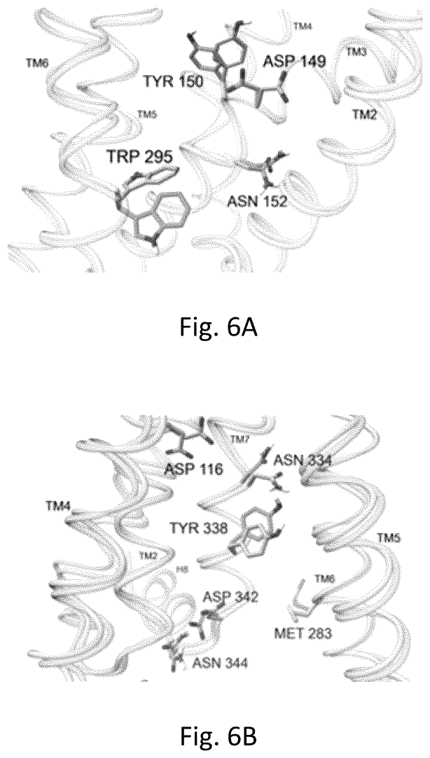

Activation is associated with rearrangement of helices in the intracellular pocket (FIG. 5A). Ballesteros-Weinstein nomenclature is used in which superscripts describe transmembrane helix (TM) or intracellular loop (ICL), followed by the position relative to the most conserved residue. In structures that favor activation, the intracellular end of TM5 is bent closer to TM1, such that the G84^{1.46}-R260^{5.64} distance can be reduced by 2 to 4 Å. TM6 is pushed outwards away from TM1, increasing the V91^{1.53}-F291^{6.30} and T99^{ICL1}-F291^{6.31} distances. A kink above a proline in TM7 becomes stronger such that the Y328^{7.43}-E343^{H8} distance is reduced. Compared to β-arrestin-2 activation, G protein activation is favored when TM5 and TM6 are bent further outward relative to helix 8 (FIG. 5B).

FIGS. 5A-B illustrate an example of alpha carbon distances with the largest (5A) general and (5B) selective activation scores. Structures are medoids of conformations from the machine learning model, colored by different types of activity: high (conformation 1), low (conformation 14), G protein (conformation 4), and β-arrestin-2 (conformation 2). They are shown from the membrane perspective facing the intracellular pocket. Dashed lines between atoms are shown for the top 2% of activation function scores.

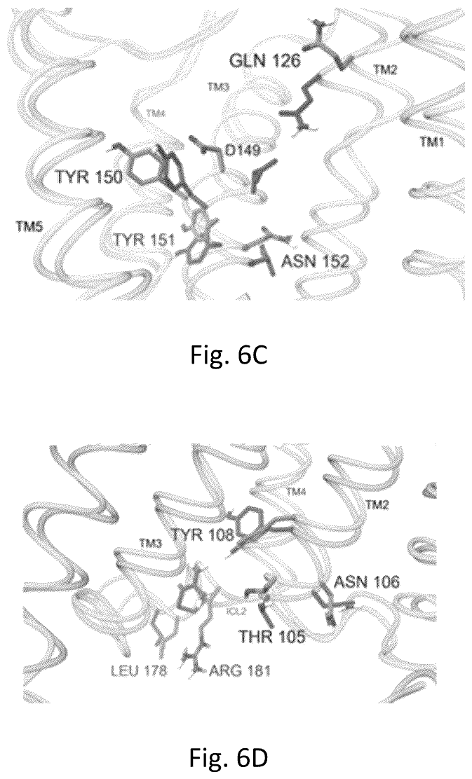

Activation is also associated with side chain dihedral angles of several amino acid residues in the orthosteric binding pocket (FIG. 6). In W295^{6.48}, activation relates to a shift in the χ₂angle from around −120° to around 120°, rotating the indole ring towards the intracellular pocket. Across the binding pocket, D149^{3.32}, Y150^{3.33}, and N152^{3.35} have distinct active and inactive conformations. In active conformations, the carboxylate of D149^{3.32} and the phenol of Y150^{3.33} extend across the binding pocket towards TM5 and TM6. In contrast, D149^{3.32} tucks towards TM2 and Y150^{3.33} extends back towards TM4 in inactive conformations. The orientation of N152^{3.35} coincides with D149^{3.32} and Y150^{3.33}, pointing the side chain amide further away from the center of the receptor in active conformations (FIG. 6A). D149^{3.32} and Y150^{3.33} also have high selective activation scores; in G protein selective conformations, the side chains of these residues are further oriented towards a sub pocket of the binding site between TM3, TM4, and TM5 (FIG. 6C).

FIGS. 6A-D illustrate examples of amino acid positions containing dihedral angles with the highest general and selective activation scores. Structures are medoids of conformations from the machine learning model, colored by different types of activity: high (conformation 1), low (conformation 14), G protein (conformation 4), and β-arrestin-2 (conformation 2). Displayed amino acids contain dihedral angles in the top two percent of general (6A, 6B) activation scores or (6C, 6D) selective activation scores. Side chain rotations were adjusted to match the degree with the highest value of the (6A, 6B) general or (6C, 6D) selective activation function. Views of the binding pocket (6A, 6C) are from the perspective behind the binding pocket region of TM7. TM1 and TM7 (6A) and TM6 and TM7 (6C) were removed to improve the display of binding pocket amino acids. Views of the intracellular region are from the perspective behind TM3 (6B) and TM7 (6D). TM3 (6B), TM6 and TM7 (6D) are removed to clearly display the intracellular amino acids.

Several residues in the interior of the intracellular pocket also have distinct orientations in active and inactive conformations (FIG. 6). D116^{2.50} extends down towards intracellular space in active conformations opposed to upwards towards interhelical space in inactive conformations. Along the interior of intracellular TM7, N334^{7.49} and Y338^{7.53} are rotated upwards in active compared to inactive conformations. At the intracellular interface, polar residues M283^{6.23}, D342^{H8}, and N344^{H8} assume different side chain configurations in active and inactive conformations.

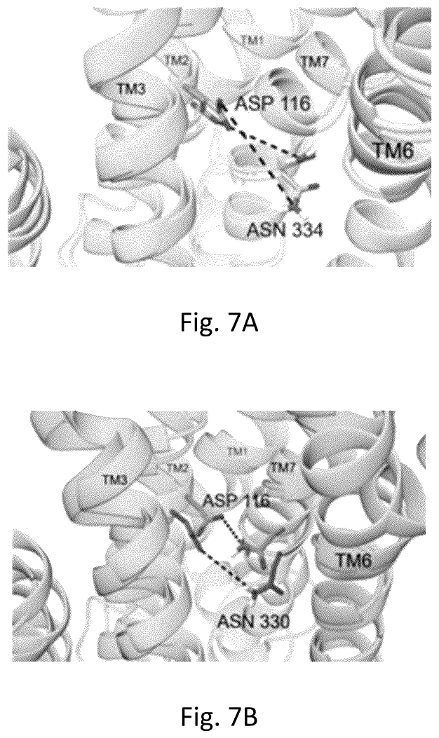

While intracellular pocket side chain dihedrals with large general activation scores are primarily in TM6, TM7, and H8, those with large selective activation scores are across the pocket in ICL2 and TM2 (FIG. 7). The alcohol group of T105^{2.39} and the amide group of N106^{2.40} extend towards ICL2 in G protein selective conformations but towards ICL1 in β-arrestin-2 selective conformations. Y108^{2.42} orients across the intrahelical space towards TM3 in β-arrestin-2 selective conformations but downwards towards intracellular space in G protein selective conformations. On ILC2, R181^{ICL2} reaches into intracellular space in the G protein selective conformations but is retracted into the receptor in β-arrestin-2 selective conformations, accompanied by different configurations of nearby L178^{1CL2}.

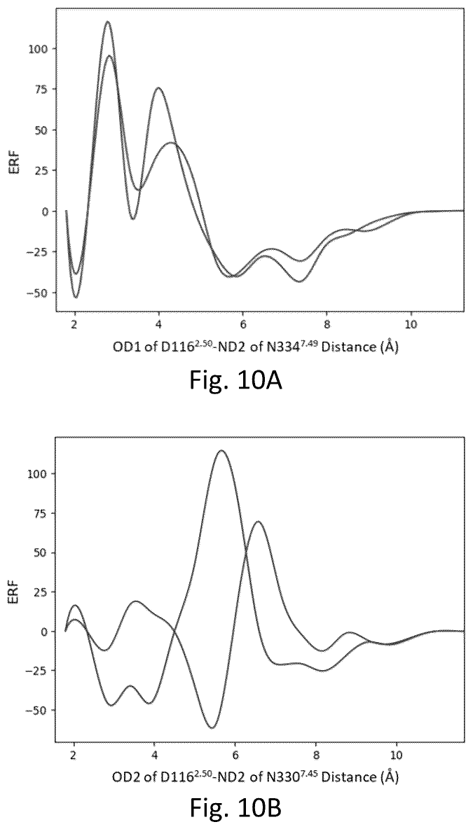

Polar networks in the sodium binding pocket are important both for general and selective activation. The network in this region includes distances between polar atoms of residues D116^{2.50}, N330^{7.45}, and N334^{7.49} (FIG. 7). Two distances of ˜3 and ˜4 Å between the first carboxylate atom (OD1) of D116^{2.50} and the nitrogen atom (ND2) of the amide side chain in N334^{7.49} are correlated with general activation. The distance between the second carboxylate atom (OD2) of D116^{2.50} and the nitrogen atom (ND2) of the amide side chain of N330^{7.45} differs in G protein (peaked around ˜5.5 Å) opposed to β-arrestin-2 signaling (peaked around ˜6.5 Å) (FIGS. 10A-B). It is also noteworthy that the neighboring residues W295^{6.48} and N152^{3.35} had dihedral angles with large general activation scores.

FIG. 7 illustrates an example of distances in the sodium binding pocket with the highest general and selective activation scores. Structures are medoids of conformations from the machine learning model. FIG. 7A shows distances between OD1 of D116^{2.50} and ND2 of N334^{7.49} in structures with high (conformation 1) and low (conformation 14) activity. FIG. 7B shows distances between OD2 of D116^{2.50} and ND2 of N330^{7.49} in conformations with high G protein (conformation 4) and β-arrestin-2 (conformation 2) activity. TM5 is removed to improve display of amino acid distances in the sodium binding pocket.

Signaling efficacy of, for example, MOR, is linearly proportional to the equilibrium population of intracellular pocket conformations. Equilibrium populations can be accurately estimated by molecular dynamics simulations. Suitable definitions of these intracellular pocket conformations can then be determined by training a machine learning model. Intracellular pocket conformations have a broad range of signaling efficacy along different pathways. Efficacy response functions and activation scores are effective metrics for identifying structural features associated with general and selective activation. These analyses lead to predicted structural mechanisms for general and selective activation that are supported by previous computational and experimental studies.

Thus, the invention provides a method and system for predicting biological signaling efficacy. The invention provides improved and efficient methods for designing drugs to modulate one or more biological signaling pathways, by predicting an efficacy of a ligand for one or more biological signaling pathways and prioritizing and/or designing ligands based on predicted efficacies.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method performed by one or more computers for predicting effects of ligands on receptors, the method comprising:
identifying a plurality of conformations of a receptor;
computing a probability of each of the plurality of conformations when the receptor is in an equilibrium complex with a ligand, to obtain a set of equilibrium probabilities; and
using the set of equilibrium probabilities to predict an effect of the ligand on the receptor.

2. The method of claim 1, wherein the effect of the ligand is to modulate biological signaling with a predicted potency and/or efficacy.

3. The method of claim 1, wherein the effect of the ligand is to modulate a rate of enzyme catalysis.

4. The method of claim 1, wherein identifying the plurality of conformations comprises performing molecular dynamics simulations of the receptor complexed with different ligands to obtain a plurality of configurations.

5. The method of claim 1, wherein identifying the plurality of conformations comprises clustering sampled receptor configurations into conformations, performed with parameters such that same conformations are observed with different ligands.

6. The method of claim 1, wherein equilibrium probabilities are calculated based on a fraction of simulations of a receptor-ligand complex in which a particular receptor conformation is sampled.

7. The method of claim 1, wherein the predicting the effect of the ligand on the receptor comprises inputting equilibrium probabilities of conformations into a machine learning model.

8. The method of claim 7, wherein the machine learning model is based on multiple linear regression.

9. The method of claim 7, wherein the machine learning model is trained and cross-validated by providing inputs and outputs for a set of ligands for which the effect has been experimentally measured.

10. The method of claim 9, wherein the training and cross-validating are performed by leave-one-out procedures, a test set comprising one sample and a training set being a remainder of set data.

11. The method of claim 10, wherein the machine learning model is trained based on a leave-one-out loss function, wherein each training set is divided into a sub-test set and a sub-training set.

12. The method of claim 11, wherein the sub-test set comprises one sample and the sub-training set comprises the remainder of data.

13. The method of claim 12, wherein the leave-one-out loss function comprises a mean square error of the sub-test set, averaged over all sub-training processes for the training set.

14. The method of claim 9, wherein the training comprises optimizing hyperparameters for clustering and parameters for machine learning to minimize a loss function.

15. The method of claim 14, wherein the hyperparameters for clustering comprises a number of hierarchical clusters, a delta value for conversion between a distance matrix and a similarity matrix conversion, and a number of conformations returned from spectral clustering and the parameters for machine learning include multiple linear regression slopes.

16. The method of claim 9, further comprising cross-validating a computational system by splitting the data into a training set and a test set and assessing a quality of effect prediction in the test set.

17. A method for designing drugs to modulate one or more biological signaling pathways, comprising: predicting an efficacy of the ligand for one or more biological signaling pathways using the method of claim 1; and prioritizing and/or designing ligands based on predicted efficacies.

18. A computational system for predicting biological signaling efficacy, comprising:
at least one data processor;
at least one storage device in combination with the at least one data processor, wherein the at least one storage devices includes coded instructions that, when executed by the at least one data processor, performs operations comprising receiving conformation data from a configurational distribution of a receptor-ligand complex; and
at least one machine learning model trained to process an equilibrium probability of each conformation in the conformation data and to generate predictions of efficacy for one or more biological signaling pathways.

19. The computational system of claim 18, wherein the conformation data are obtained through molecular dynamics simulations based on standard atomistic force fields and simulation settings mirroring experimental conditions for which a prediction is desired.

20. The computational system of claim 18, wherein the at least one storage devices further includes coded instructions for categorizing sampled receptor configurations into conformations.
